(19) United States
(12) Patent Application Publication
(10) Pub. No.: US 2017/0233787 A1
(43) Pub. Date: Aug. 17, 2017

(54) METHODS AND COMPOSITIONS FOR ANALYZING GLUCOSE-6-PHOSPHATE DEHYDROGENASE ACTIVITY IN BLOOD SAMPLES

(71) Applicant: Becton, Dickinson and Company, Franklin Lakes, NJ (US)

(72) Inventors: David Sebba, Cary, NC (US); Adam Curry, Raleigh, NC (US); Kara Birchfield, Raleigh, NC (US); Kristin Weidemaier, Raleigh, NC (US); Erin Carruthers, Raleigh, NC (US)

(21) Appl. No.: 15/501,807

(22) PCT Filed: Aug. 4, 2015
(86) PCT No.: PCT/US15/43678

§ 371 Date: Feb. 3, 2017

Related U.S. Application Data
(60) Provisional application No. 62/033,548, filed on Aug. 5, 2014.

(51) Int. Cl. C12Q 1/32; G01N 33/569

(52) U.S. Cl. CPC C12Q 1/32; G01N 33/569; G01N 2333/904; G01N 2333/445

(57) ABSTRACT

Methods and compositions for the detection of glucose-6-phosphate dehydrogenase (G6PD) enzyme activity in blood samples are described. Some embodiments disclosed herein provide methods for detecting G6PD activity in undiluted or minimally diluted blood samples, including obtaining a blood sample, and detecting G6PD activity present in the undiluted or minimally diluted blood sample by epifluorescence. Also provided are methods for detecting G6PD activity and detecting a bloodborne microorganism as two parts of a single test.

### FIELD

The present disclosure generally relates to methods and compositions for analysis of blood samples, including for detection of glucose-6-phosphate dehydrogenase (G6PD) enzyme activity in undiluted or minimally diluted blood samples. In some embodiments, the detection of G6PD activity is performed as part of or in conjunction with diagnostic testing for one or more bloodborne microorganisms in the blood sample.

### BACKGROUND

The materials described in this section are not admitted to be prior art by inclusion in this section.

Glucose-6-phosphate dehydrogenase (“G6PD” or “G6PDH”) performs a critical function in cellular biochemistry. It is part of the oxidative pentose pathway, in which it functions to minimize oxidative attacks of free radicals upon cells by providing reducing equivalents. G6PD enzyme converts glucose-6-phosphate to 6-phosphoglutonate, thereby liberating a proton that reduces nicotinamide adenine dinucleotide phosphate, NAPD⁺, to NAPDH. The NAPDH initiates a series of downstream reactions that ultimately reduce the free radical oxidizing agents and render many of them ineffective in cellular biochemistry.

In humans, G6PD enzyme is present in all cell types, but it is present in higher concentration in red blood cells which, in one of their primary functions, act as oxygen transport vehicles and are hence particularly susceptible to oxidative attack. High G6PD concentrations observed in red blood cells are partly because the G6PD system is utilized in combating and preventing undesirable oxidative effects. However, when strong oxidizing agents, such as members of the quinoline class of anti-malarial drugs, including the 8-aminoquinoline class of drugs, are introduced to humans as part of malarial treatment, the need for rapid production of reducing agents is greatly increased.

G6PD deficiency is reported to be among the most common human enzyme defects, affecting more than 400 million people worldwide. In these G6PD deficient individuals, G6PD enzyme shows greatly reduced specific activity. As a result, administration of strong oxidizing agents, such as members of the class of quinoline-type anti-malarial drugs, may cause severe clinical complications, such as hemolytic anemia, because the low specific activity of their G6DP does not enable the production of sufficient reducing agents to prevent rapid unwanted oxidative effects on their red blood cells. Therefore, in areas where malarial infections are common and during malaria epidemics, a need exists for a rapid and efficient test that will readily distinguish persons having G6PD of low specific activity from persons whose G6PD activity is normal and will enable medical personnel to ensure that (1) the anti-malarial drugs which cause oxidative stress are prescribed only for individuals with normal or better G6PD specific activity, (2) individuals with mild deficiencies in G6PD activity are prescribed with a reduced dosage level of quinoline anti-malarial drugs, or alternatively, with an alternative type of anti-malarial drugs, and (3) persons with more severe G6PD activity deficiencies are medicated with an alternative type of anti-malarial drugs.

Currently available commercial tests for G6PD enzyme activity in blood samples primarily fall into two main categories; (1) those capable of providing relatively quantitative results but require expensive core-laboratory equipment and significant dilution of the blood samples, and (2) those involving measurement of chromogenic substrates that can be performed in a lateral flow format at lower cost but do not provide quantitative data. For example, G6PD enzyme activity in blood samples can be detected by measuring the intrinsic absorbance or fluorescence of the coenzymes, NADP³⁰/NADPH, or by monitoring a chromogenic dye whose color in the sample changes as a function of the concentration of NADPH. One significant disadvantage of these approaches is that they typically require the blood samples to undergo significant dilution prior to the assay in order to reduce the solution's optical density to a range that can be measured using standard spectrometers and optical equipment or can be detected by the human eye. Another disadvantage of these approaches is that the dilution process is time consuming, and involves additional handling of a potentially infectious blood sample. In addition, significantly diluting the whole blood sample often increases the error probability within the sample data.

Another problem with the currently available approaches for measuring G6PD enzyme activity in blood samples is that they typically require calibration and/or normalization by a separate measurement of hemoglobin content in the same blood samples. This is because most of the dilutions and measurements are relative rather than absolute and, therefore, in order to provide exact quantitation of the actual enzyme activity, generally stabilized samples of whole blood must be analyzed by the instruments and the results must then be adjusted so that the values are normalized relative to the concentration of a component within the blood. This process is prone to errors in the preparation of the standard material and its stability during transportation and storage.

An alternative approach for measuring G6PD enzyme activity in undiluted blood samples involves the use an extremely short path length cuvette or instrumentation capable of measuring absorbance of high optical density samples. However, this approach has not been deployed in G6PD commercial tests due to the prohibitively high costs of ultra-short-pathlength cuvettes and/or instrumentation that are typically required for use with concentrated samples in order to accurately measure the optical density of the sample, which would significantly increase the cost of the commercial tests.

As mentioned above, because malaria infection and a number of anti-malarial drugs have been widely reported to induce oxidative stress in blood cells, particularly in the parasitized erythrocytes, testing of G6PD enzyme activity in blood samples is often performed in conjunction with diagnostic testing for Plasmodium sp. microorganisms, the most common causative agents of malaria. This however, until presently, requires two separate tests: one for malaria, and another for G6PD enzyme activity.

Thus, there is a need in the art for a point-of care test that integrates diagnosis of febrile illness, including malaria, with a quantitative G6PD test. This is because if an individual is malaria positive, G6PD enzyme activity status is required to determine the appropriate course of treatment. An integrated test may provide a number of advantages, including avoiding the cost of an additional diagnostic test, avoiding the need to obtain an additional blood sample, and better workflow. This ensures that the appropriate course of treatment can be determined at the time of diagnosis when medicine is prescribed. Additionally, there is a problem in the art that sensitive diagnosis of malaria requires a minimally diluted blood sample to avoid diluting the pathogen, while quantitative G6PD tests require a highly diluted blood samples to avoid problems of measuring signals from high optical density samples using analytical instruments. These two requirements would be in conflict when the two tests are combined into a single test.

In one aspect, the present application discloses compositions and methods useful for performing quantitative G6PD testing on minimally diluted blood samples, optionally as part of or in conjunction with diagnostic testing for one or more bloodborne microorganism, for example Plasmodium sp. microorganisms. In some particular embodiments, the G6PD testing methods disclosed herein are particularly useful for integrating a quantitative G6PD test into a multiplexed diagnostic workflow for detecting bloodborne microorganism, such as causative microorganism of malaria and other febrile diseases.

### SUMMARY

This section provides a general summary of the disclosure, and is not comprehensive of its full scope or all of its features.

Disclosed herein are methods for detecting glucose-6-phosphate dehydrogenase (G6PD) activity that includes obtaining or receiving a diluted or a minimally diluted blood sample and detecting G6PD activity present in the undiluted or minimally diluted blood sample. In some embodiments the undiluted or minimally diluted blood sample is obtained from a subject. In some embodiments of the methods, the detecting G6PD activity includes performing epifluorescence detection on said undiluted or minimally diluted blood sample.

Implementations of methods according to the disclosure can include one or more of the following features. In some embodiments, the detecting G6PD activity includes measuring a signal corresponding to the enzymatic conversion of β-nicotinamide adenine dinucleotide 2′-phosphate (“NADP”) to β-nicotinamide adenine dinucleotide 2′-phosphate, reduced (“NADPH”) in the undiluted or minimally diluted blood sample. In some embodiments, the conversion of NADP+ to NADPH is measured by monitoring the fluorescence of a dye molecule which interacts with NADPH. In some embodiments, the detecting G6PD activity includes measuring NADPH fluorescence. In some embodiments, the NADPH fluorescence is spectrophotometrically performed via measurement of the NADPH emission when excited by ultraviolet light. In some embodiments, the NADPH fluorescence is excited at a wavelength or wavelengths between 290-400 nm. In some embodiments, the NADPH fluorescence is excited at a wavelength or wavelengths between 310-380 nm. In some embodiments, the NADPH fluorescence is excited at a wavelength or wavelengths between 330-370 nm. In some embodiments, the measuring of NADPH fluorescence is performed at 365 nm excitation. In some embodiments of the methods disclosed herein, the detecting G6PD activity is performed via an Attenuated Total Reflectance (ATR) approach. In some embodiments of the methods disclosed herein, the detecting G6PD activity includes measuring a signal corresponding to the conversion of glucose-6-phosphate (G6P) to 6-phosphoglucono-lactone in the undiluted or minimally diluted blood sample. In some embodiments, the detecting G6PD activity is not substantially affected by fluctuations in temperature. In some embodiments, the detecting G6PD activity is not substantially affected by fluctuations in blood concentration in the reaction.

Some embodiments disclosed herein relate to methods for detecting G6PD enzyme activity in an undiluted or minimally diluted blood sample in which the detection of G6PD activity in the undiluted or minimally diluted blood sample is performed as part of or in conjunction with a diagnostic method for detecting a bloodborne microorganism in the blood sample. In some embodiments, the detection of G6PD activity and detection of a bloodborne microorganism are performed on the same aliquot of undiluted or minimally diluted blood sample. In some embodiments, the bloodborne microorganism is selected from the group consisting of a bacterium, a protozoan, a mold, a yeast, a filamentous microfungus, and a virus. In some embodiments, the bloodborne microorganism is a causative microorganism of malaria. In some embodiments, the causative microorganism of malaria is a microorganism belonging to a protozoan genus selected from the group consisting of Plasmodium, Polychromophilus, Rayella, and Saurocytozoon. In some embodiments, the causative microorganism of malaria is a Plasmodium microorganism belonging to a subgenus selected from the group consisting of Asiamoeba, Bennettinia, Carinamoeba, Giovannolaia, Haemamoeba, Huffia, Lacertamoeba, Laverania, Novyella, Paraplasmodium, Plasmodium, Sauramoeba, and Vinckeia. In some embodiments, the causative microorganism of malaria is a Plasmodium microorganism selected from the group consisting of Plasmodium falciparum, Plasmodium knowlesi, Plasmodium malariae, Plasmodium ovale, and Plasmodium vivax.

In some embodiments of the methods disclosed herein, the detection of a bloodborne microorganism in the undiluted or minimally diluted blood sample includes determining the level, or the presence, of at least one biomarker specific to the bloodborne microorganism. In some embodiments, the at least one biomarker is an antigen specific to the bloodborne microorganism. In some embodiments, the antigen is selected from the group consisting of aldolase (pFBPA), histidine-rich protein 2 (HRP-2), hypoxanthine phosphoribosyltransferase (pHPRT), lactate dehydrogenase (pLDH), and phosphoglycerate mutase (pPGM). In some embodiments, the detection of a bloodborne microorganism is carried out by an immunoassay. In some embodiments, the detection of a bloodborne microorganism is carried out by a sandwich immunoassay. In some embodiments, the immunoassay for detection of a bloodborne microorganism is carried out by a microparticle-based SERS nanotag immunoassay. In some embodiments, the microparticle-based SERS nanotag immunoassay for detection of the bloodborne microorganism is a homogenous immunoassay. The detection of the bloodborne microorganism, in various embodiments of the present disclosure, can be carried out by an Enzyme Linked Immunosorbent Assay (ELISA), or other sandwich immunoassays such as bead-based immunoassays.

In some embodiments, the detection of G6PD activity and detection of a bloodborne microorganism are carried out simultaneously on a single reaction mixture. In some embodiments, the detection of G6PD activity and detection of a bloodborne microorganism are carried out sequentially on a single reaction mixture. In some embodiments, the undiluted or minimally diluted blood sample is divided into sample aliquots prior to being subjected to the detection of G6PD activity and detection of a bloodborne microorganism. In some embodiments, the detection of G6PD activity and detection of a bloodborne microorganism are carried out in spatially discrete sample aliquots.

Provided herein are kits for detecting an amount of glucose-6-phosphate dehydrogenase (G6PD) activity in a undiluted or minimally diluted blood sample, which includes (a) glucose-6-phosphate (G6P) or an G6P surrogate adapted for use in an undiluted or a minimally diluted blood sample; (b) nicotinamide adenine dinucleotide phosphate (NADP+) or NADP+ surrogate adapted for use in an undiluted or a minimally diluted blood sample. In some embodiments, the kits provided herein further include instructions for preparing a reaction mixture that facilitates a reaction of NADP+, G6P, and G6PD enzyme in an undiluted or minimally diluted blood sample. In some embodiments, the kits further include immunoassay reagents for detection of a bloodborne microorganism. In some embodiments, the undiluted or minimally diluted blood sample is from a subject. In some embodiments, the subject is suffering from, or suspected of suffering, from a disease. In some embodiments, the subject is suffering from, or suspected of suffering, from a febrile illness. In some embodiments, the disease is caused by a bloodborne microorganism. In some embodiments, the disease is malaria.

In some embodiments of the method or kits disclosed herein, in the blood sample subject to analysis the concentration of whole blood or a blood component in the final reaction mixture is greater than 0.1%. In some embodiments, in the blood sample subject to analysis the concentration of whole blood or a blood component in the final reaction mixture is greater than 1%. In some embodiments, in the blood sample subject to analysis the concentration of whole blood or a blood component in the final reaction mixture is greater than greater than 10%, greater than 20%, greater than 25% greater than 50%, greater than 75%, greater than 80%, greater than 90%, greater than 99%, and optionally not greater than 80%, 90% or 99%. In some embodiments, in the blood sample subject to analysis the concentration of whole blood or a blood component in the final reaction mixture is from 0.1% to 95%, 1% to 95%, 1% to 68%, 25% to 99%, 25% to 95%, 25% to 70%, 40% to 80%, 40% to 68%, 50% to 95%, 50% to 68%, 60% to 95%, 60% to 80%, 68% to 95%, or 68% to 90%. In some embodiments of the method or kits disclosed herein, in the blood sample subject to analysis, the concentration of whole blood or a blood component in the final reaction mixture is undiluted, or minimally diluted.

The foregoing summary is illustrative only and is not intended to be in any way limiting. In addition to the illustrative embodiments and features described herein, further aspects, embodiments, objects and features of the disclosure will become fully apparent from the drawings and the detailed description and the claims.

### BRIEF DESCRIPTION OF THE DRAWINGS

FIG. 1 illustrates an example of a process of reducing NADP⁺ to NADPH to generate a fluorescence signal.

FIG. 2 illustrates the results from an embodiment of an integrated assay for detecting G6PD enzymatic activity in conjunction with a diagnostic assay of a bloodborne parasitic microorganism in undiluted blood samples. Undiluted human blood hemolysate normal or deficient G6PD activity controls (Trinity Biotech) were spiked with 0 ng/mL or 150 ng/mL of a lactate dehydrogenase (pLDH) antigen of Plasmodium vivax, a parasitic protozoan and causative agent of malaria in human. Levels of G6PD enzymatic activity (solid bars) were assayed in the presence (150 ng/mL; Vivax+) or absence (0 ng/mL; Vivax−) of the pLDH) antigen. Malaria detection assays (dashed bars) were performed using a SERS nanotag immunoassay in which immunoassay reagents were conjugated to either a capture antibody panspecific pLDH antibody or a detector antibody specific to the P. vivax pLDH antigen. “a.u” stands for Arbitrary Unit. The blood concentration of was 9% (i.e. 90 μL of blood per 1000 μL total assay volume) in all samples.

FIG. 3 graphically summarizes the results of embodiments of experiments measuring levels of G6PD enzymatic activity in blood samples (9%) based on the absorption of NADPH at 315 nm, making use of the spectrophotometric change when NADPH is produced. Undiluted blood samples from G6PD normal (G6PD Normal) or G6PD deficient (G6PD Def.) human blood hemolysate control samples (Trinity Biotech) were spiked with 0 ng/mL or 150 ng/mL of a lactate dehydrogenase (pLDH) antigen of Plasmodium vivax. Levels of G6PD enzymatic activity were assayed in the presence (150 ng/mL; Malaria+) or absence (0 ng/mL; Malaria−) of the pLDH antigen. The 315 nm absorbance was monitored for 5 minutes, and the change in OD over this time period was calculated.

FIG. 4 graphically summarizes the results of embodiments of experiments measuring levels of G6PD enzymatic activity by epifluorescence spectrometry at two exemplary blood concentrations. The plots show epifluorescence G6PD enzymatic data of normal and deficient activity hemolysate blood control samples. Assays using the hemolyzed blood controls were collected at 0.33% blood and 9.0% blood, respectively. Reagents concentrations were scaled proportionally with the blood concentrations.

FIG. 5 graphically illustrates the results of embodiments of experiments measuring levels of G6PD enzymatic activity by epifluorescence spectrometry at 68% blood concentration. Normal, Intermediate, and Deficient activity hemolyzed blood controls were tested at 68% blood using the epifluorescence method, and enzyme activity was able to be distinguished. Assay reagent concentrations were increased to ensure that enzyme activity rather than reagent concentration was the limiting factor for the reduction of NADP+ to NADPH.

FIG. 6 graphically illustrates the results of embodiments of experiments measuring levels of G6PD enzymatic activity by epifluorescence spectrometry at two exemplary temperatures. The experiments presented in FIGS. 6 were performed at 25° C. and 40° C. while the experiments presented at FIG. 4 above were performed at 18° C.

FIGS. 7A and 7B summarize the results of embodiments of experiments measuring levels of G6PD enzymatic activity in 17 clinical blood samples. G6PD activity was assayed either by an epifluorescence spectrometry test at a concentration of 9% blood in the assay solution according to the methods disclose herein (FIG. 7A) or by a commercial Trinity quantitative test (FIG. 7B). In this experiment, three G6PD enzymatic activity controls were also included (Deficient, Intermediate, and Normal). Negative values observed in the epifluorescence data were determined to be due to photobleaching of the plastic cuvettes used for testing, rather than a biological phenomenon.

FIG. 8 is a graphical representation of embodiments of experiments of Trinity quantitative test and epifluorescence test concordance from testing of 154 undiluted blood samples. Solid triangles: >40% enzyme activity. Solid circles: 40-70% enzyme activity. Solid squares: >70% enzyme activity. Negative values observed in the epifluorescence data are due to photobleaching of the plastic cuvettes used for testing, rather than a biological phenomenon. In the Trinity quantitative tests, the G6PD activity was normalized relative to the reported mean enzyme activity of a healthy adult male, which was 7.17 IU/g Hb.

FIG. 9 is a graphical representation of embodiments of experiments of Trinity quantitative test and epifluorescence test concordance from testing of 154 undiluted blood samples. Each of the samples was assayed for G6PD activity by (1) Trinity quantitative test and (2) epifluorescence test. Data was analyzed according to following four protocols. Top Left: Neither Trinity quantitative test nor epifluorescence test was normalized by a separate hemoglobin measurement; Pearson Correlation Coefficient=0.896. Top Right: Trinity quantitative test was normalized by a separate hemoglobin measurement, while epifluorescence test was not normalized by a separate hemoglobin measurement; Pearson Correlation Coefficient=0.968. Bottom Left: Trinity quantitative test was not normalized by a separate hemoglobin measurement, while epifluorescence was test normalized by a separate hemoglobin measurement; Pearson Correlation Coefficient=0.671. Bottom Right: both Trinity quantitative test and epifluorescence test were normalized by a separate hemoglobin measurement; Pearson Correlation Coefficient=0.893 (Pearson Correlation Coefficient=0.935 with single outlier at 11.73 IU/g Hb removed). Negative values observed in the epifluorescence data were determined to be due to photobleaching of the plastic cuvettes used for testing, rather than a biological phenomenon.

### DETAILED DESCRIPTION

The present disclosure generally relates to methods, compositions and kits for analysis of blood samples. In some embodiments, the disclosure particularly relates to methods, compositions, and kits useful for the determination of glucose-6-phosphate dehydrogenase (G6PD) enzyme activity as part of or in conjunction of an immunoassay test for the presence of a bloodborne microorganism in a blood sample.

In the following detailed description, reference is made to the accompanying Figures, which form a part hereof. The illustrative embodiments described in the detailed description, Figures, and claims are not meant to be limiting. Other embodiments may be used, and other changes may be made, without departing from the spirit or scope of the subject matter presented here. It will be readily understood that the embodiments of the present disclosure, as generally described herein, and illustrated in the Figures, can be arranged, substituted, combined, and designed in a wide variety of different configurations, all of which are explicitly contemplated and make part of this disclosure.

Unless expressly defined otherwise, all terms of art, notations and other scientific terms or terminology used herein are intended to have the meanings commonly understood by those of skill in the art to which this disclosure pertains when read in light of this disclosure.

### A. Some Definitions

The singular form “a”, “an”, and “the” include plural references unless the context clearly dictates otherwise. For example, the term “a cell” includes one or more cells, including mixtures thereof. As used herein, the term “and/or” includes any and all combinations of one or more of the associated listed items. Thus, “A and/or B” is used herein to include all of the following embodiments: “A”, “B”, “A or B”, and “A and B”. It will be further understood that the terms such as “comprises”, “comprising”, “includes” and/or “including”, when used herein, specify the presence of stated features, integers, steps, operations, elements, and/or components, but do not preclude the presence or addition of one or more other features, integers, steps, operations, elements, components, and/or groups thereof. Therefore, said terms encompass the terms “consisting essentially of” and “consisting of”.

“About” has its ordinary meaning of approximately. If the degree of approximation is not otherwise clear from the context, “about” means either within plus or minus 10% of the provided value, or rounded to the nearest significant figure, in all cases inclusive of the provided value. Where ranges are provided, they are inclusive of the boundary values.

As used, herein, the term “antigen” refers to a protein, glycoprotein, lipoprotein, lipid or other substance that is reactive with an antibody specific for a portion of the molecule.

The terms “biological sample” and “test sample” refer to all biological fluids and excretions isolated from any given subject or subjects. In the context of the present disclosure such samples include, but are not limited to, blood, blood serum, blood plasma, nipple aspirate, urine, semen, seminal fluid, seminal plasma, prostatic fluid, excreta, tears, saliva, sweat, biopsy, ascites, cerebrospinal fluid, milk, lymph, bronchial and other lavage samples, or tissue extract samples. As used herein, “blood sample” encompasses whole blood, plasma or serum. Typically, whole blood is the preferred test sample for use in the context of the present disclosure.

As used herein, “diluent” refers to a component added to the sample for the sole purpose of diluting the sample, and does not refer to a reagent added to the mixture for analysis of the sample (e.g. an antibody, glucose-6-phosphate (G6P), nicotinamide adenine dinucleotide phosphate (NADP+), etc.).

The phrase “minimally diluted”, as used herein, encompasses a blood sample which is either not diluted at all (i.e. undiluted) or has not been significantly diluted. “Minimally diluted” does encompass a sample that has had one or more reagents added to the sample for purposes of the analysis, other than to dilute the sample. For example, in some embodiments, a minimally diluted sample has a solution comprising glucose-6-phosphate (G6P), nicotinamide adenine dinucleotide phosphate (NADP^{±}), or immunoassay reagents including antibodies or antibody conjugates added to the sample. Non-limiting examples of reagents added to the sample for purposes of the analysis, other than to dilute the sample, include buffer components with utility such as cell lysis, pH buffering, stabilizing reagents, anticoagulants, blocking undesirable nonspecific binding, etc. To the extent the addition of the reagents dilutes the sample, if at all, such dilution has no significant impact on the analysis performed when the sample is “minimally diluted. Accordingly, in some embodiments, reagents used in the methods and compositions disclosed herein include anticoagulants (for example, EDTA, heparin) and in some instances an isovolumetric sphering agent, or an aggregating agent.

Moreover, under certain circumstances (for example, very rapid analysis), it may not be necessary to add the anticoagulating agent, but it is preferable to do so in most cases to ensure the sample is in a form acceptable for analysis. In this regard, as one skilled in the art will readily appreciate, although whole blood samples used in the methods disclosed herein can be undiluted or minimally diluted, if desired, some degree of dilution could occur. This is because in the practice of the methods, it may be desirable to add reagents such as anticoagulant in a liquid rather than a solid format. Anticoagulated whole blood as described herein can be produced by adding anticoagulants to whole blood either prior to the addition of whole blood to the reaction mixture or anticoagulant can be added to the reaction mixture either before or after addition of the blood to the reaction mixture.

Regardless of the number of reagents added for analysis, as used herein “minimally diluted” means that the final concentration of whole blood or a blood component in the final reaction mixture which is subject to analysis is greater than 0.1%, greater than 1%, greater than 5%, greater than 10%, preferably greater than 20%. In some embodiments, final concentration of the minimally diluted blood sample is greater than 20%, greater than 25%, greater than 30%, preferably greater than 35%, preferably greater than 55%, preferably greater than 60%, preferably greater than 65%. In some embodiments, final concentration of the minimally diluted blood sample is greater than 60%, greater than 65%, greater than 70%, preferably greater than 75%, preferably greater than 80%, preferably greater than 85%, preferably greater than 90%. In some embodiments, final concentration of the minimally diluted blood sample is greater than 95%, 98%, 99%, or is 100% (undiluted). In any of these embodiments, the final concentration can also be not greater than 50%, 60%, 70%, 75%, 80%, 85%, 90% or 99%. Thus, in some embodiments the final concentration is within a range defined by any the preceding lower and upper limits. For example, in some embodiments the final concentration is from 0.1% to 99%, from 50% to 99%, from 50% to 80%, from 50% to 70%, from 60% to 99%, from 60% to 80%, from 70% to 95%, etc.

While use of an undiluted or minimally diluted blood sample is preferred, it is also contemplated that a diluted blood sample can be used. Thus, it is contemplated that in some embodiments of the methods, compositions and kits disclosed herein, the “undiluted” or “minimally diluted” blood sample can instead be a blood sample which is diluted by a suitable diluent such that the concentration of whole blood or a blood component in the final reaction mixture which is subject to analysis is less than 80%, 50%, 25%, 5%, 1%, or 0.1%.

As used herein, the terms “epifluorescence detection,” “epifluorescence spectroscopy,” “epifluorescence spectrometry,” and “epifluorescence illumination”, refer to fluorescence detection techniques where both the illumination (also referred to as “excitation light”) and emitted light travel through the same objective lens. In some embodiments, the light source can be mounted relative to the sample specimen such that the excitation light passes through the objective lens on its way toward the specimen and the emitted light passes through the objective lens on its way towards the detector. In some embodiments, the excitation light is excluded from reaching the detector by a filter and/or dichroic mirror which reflects or absorbs the excitation light but transmits the emission light to the detector. In some embodiments, a modified epifluorescence-like configuration is used in which the excitation light and emitted light pass through two separate objective lenses.

The term “malaria” refers to the art recognized infectious disease found in a number of animal subjects, including birds, reptile, human and non-human primates, known as “malaria” disorders which are caused by a protozoan of the genera Plasmodium, Fallisia, or Saurocytozoon. There are over 100 malaria causative microbial species of which 22 infect non-human primates and 82 are pathogenic for reptiles and birds. In humans, the term malaria is often used interchangeably with ague or marsh fever to refer to infectious diseases typically caused by a parasite of the genus Plasmodium, such as P. falciparum, P. knowlesi, P. malariae, P. ovale, or P. vivax. This parasite is typically transmitted by female Anopheles mosquitoes, by infected blood transfusions, or transplacentally. Plasmodium parasites invade and consume the red blood cells of its hosts, which leads to symptoms including fever, anemia, and in severe cases, a coma potentially leading to death.

The term “malaria diagnostic antibody” refers to any antibody capable of binding to a protein specifically related to a malaria parasite infection, for example, an anti-pLDH antibody specifically binds to a Plasmodium lactate dehydrogenase (LDH) polypeptide.

The term “microorganism” as used herein has its conventional meaning in the art and includes, but is not limited to bacteria, filamentous microfungi, protozoa, yeasts, molds, and viruses. A “bloodborne microorganism”, as used herein, is intended to encompass any microorganism that can be found in blood. As such, the term “bloodborne microorganism” encompasses bloodborne pathogens that can cause disease in humans. Bloodborne pathogens can cause disease when transferred from an infected person to another person through blood or other potentially infected body fluids. Bloodborne pathogens thus include, but are not limited to, hepatitis B virus (HBV), hepatitis C virus (HCV), human immunodeficiency virus (HIV), West Nile virus, causative microorganisms of malaria, and syphilis.

The term “signal” as used herein can refer to any detectable parameter. Examples of these parameters include optical, electrical, or magnetic parameters, current, fluorescent emissions, infrared emissions, chemiluminescent emissions, ultraviolet emissions, light emissions, and absorbance of any of the foregoing. A signal, for example, may be expressed in terms of intensity versus distance along a diagnostic lane of an assay device. In another example, a signal may be expressed in terms of intensity, or intensity versus time. The term “signal” can also refer to the lack of a detectable physical parameter.

As used herein, the term “subject” refers to animals, including mammals, preferably humans, that is the source of a blood sample assayed in accordance with the methods described herein. The term “mammal”, according to some embodiments of the methods disclosed herein, includes both humans and non-humans and include but is not limited to humans, non-human primates, canines, felines, murines, bovines, equines, and porcines. As such, as used herein, animals can include domestic and farm animals, zoo animals, sports or pet animals, such as birds, dogs, horses, cats, cattle, pigs, sheep, etc. In some embodiments, the term subject refers to domesticated non-mammal animals with canines, felines, fowl, poultry, and small reptiles being the most preferred. In some embodiments, the undiluted blood samples are sourced preferably from mammals, most preferably from human subjects.

As will be understood by one skilled in the art, for any and all purposes, such as in terms of providing a written description, all ranges disclosed herein also encompass any and all possible sub-ranges and combinations of sub-ranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third. As will also be understood by one skilled in the art all language such as “up to,” “at least,” “greater than,” “less than,” and the like include the number recited and refer to ranges which can be subsequently broken down into sub-ranges as discussed above. Finally, as will be understood by one skilled in the art, a range includes each individual member. Thus, for example, a range of 1-3 refers to at least the values 1, 2, or 3. Similarly, for example a group having 1-5 articles refers to groups having 1, 2, 3, 4, or 5 articles, and so forth.

B. Detecting Glucose-6-phosphate Dehydrogenase Activity in Blood Samples

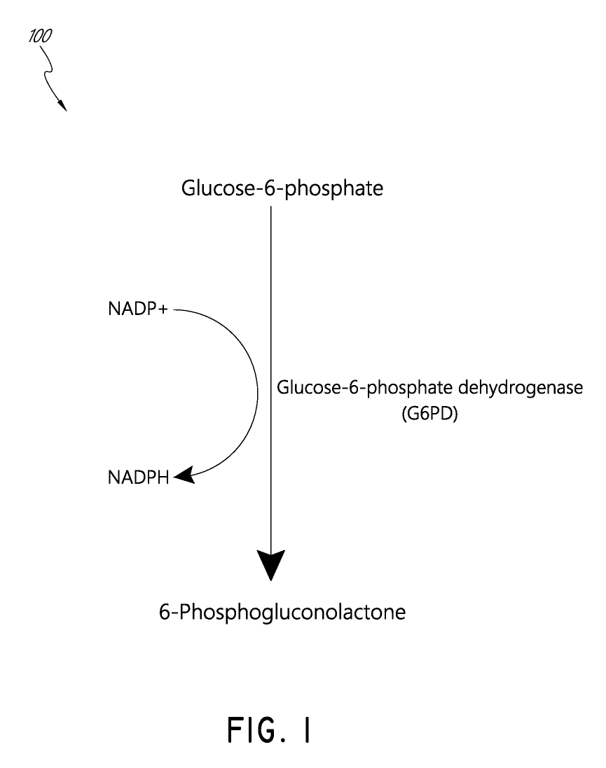

The G6PD enzyme catalyzes the oxidation of glucose-6-phosphate to 6-phosphoglucolactone while concomitantly reducing the oxidized form of nicotinamide adenine dinucleotide phosphate (NADP⁺) to nicotinamide adenine dinucleotide phosphate (NADPH). The NADPH produced will fluoresce under longwave UV light (for example 340 nm excitation/460 nm emission) during the reaction. FIG. 1 illustrates an example of a process 100 of reducing NADP⁺ to NADPH to generate a fluorescence signal. As glucose-6-phosphate is oxidized to 6-phosphogluconolactone, the coenzyme NADP⁺ is reduced to NADPH with a corresponding elevation in fluorescence.

A particular advantage of the presently disclosed methods is that the methods enable measurement of glucose-6-phosphate dehydrogenase (G6PD) activity in a sample, especially in a whole blood sample which is not diluted or which is minimally diluted, within a range of maximum 1000 times, especially less or equal to 2 times.

Accordingly, in some embodiments, the present disclosure provides a method for detecting glucose-6-phosphate dehydrogenase (G6PD) activity in undiluted or minimally diluted blood samples. Such a method includes obtaining or receiving an undiluted or a minimally diluted blood sample, for example from a subject, nurse, physician or lab technician, and detecting G6PD activity present in the undiluted or minimally diluted blood sample. In such a method, the detecting G6PD activity includes performing epifluorescence spectroscopy on the undiluted or minimally diluted blood sample in order to measure the rate at which the G6PD enzyme reduces NADP+ to NADPH.

In some embodiments, to assay G6PD activity, the whole blood sample can be diluted by a blood/diluent ratio more than 1:1000, more than 1:100, more than 1:20; more than 1:10; more than 1:5; more than 1:1; more than 2:1; more than 3:1; preferably more than 4:1; preferably more than 5:1; preferably more than 10:1; preferably more than 25:1; more preferably more than 50:1; more preferably more than 80:1; more preferably more than 90:1; and most preferably more than 100:1. In some embodiments, to assay G6PD activity, the blood sample can be diluted by a blood/diluent ratio within a range of about 100:1 to 75:1. In some embodiments, to assay G6PD activity, the whole blood sample can be diluted by a blood/diluent ratio within a range of about 1:20 to 1:1. In some embodiments, to assay G6PD activity, the whole blood sample can be diluted by a blood/diluent ratio within a range of about 1:1 to 10:1. In some embodiments, to assay G6PD activity, the whole blood sample can be diluted by a blood/diluent ratio within a range of about 10:1 to 25:1. In some embodiments, to assay G6PD activity, the whole blood sample can be diluted by a blood/diluent ratio within a range of about 20:1 to 75:1. In some embodiments, to assay G6PD activity, the blood sample can be diluted by a blood/diluent ratio within a range of about 75:1 to 100:1. In some embodiments, to assay G6PD activity, the blood sample can be diluted by a blood/diluent ratio within a range of about 50:1 to 100:1. In some embodiments, to assay G6PD activity, the blood sample can be diluted by a blood/diluent ratio within a range of about 25:1 to 100:1. In some embodiments, to assay G6PD activity, the blood sample can be diluted by a blood/diluent ratio within a range of about 20:1 to 100:1. In some embodiments, to assay G6PD activity, the blood sample can be diluted by a blood/diluent ratio within a range of about 10:1 to 100:1. In some embodiments, to assay G6PD activity, the blood sample can be diluted by a blood/diluent ratio within a range of about 10:1 to 75:1. In some embodiments, to assay G6PD activity, the blood sample can be diluted by a blood/diluent ratio within a range of about 20:1 to 50:1. In some embodiments, the detection of G6DP activity is performed on whole blood samples that are not diluted by any diluents prior to being subjected to G6PD activity analysis.

In some embodiments, the detection of G6PD activity includes performing epifluorescence detection directly on a blood sample, preferably that is minimally diluted. Epifluorescence detection (utilizing a lens to deliver the excitation illumination to the sample and to collect the emitted fluorescence) provides a method to reduce the effective optical pathlength optically, allowing the enzyme assay to be run at the elevated blood loads that are desirable for immunoassays. Typically, a higher intensity excitation light is used to excite a fluorescent molecule in the sample thereby causing the fluorescent molecule to emit fluorescent light. The excitation light has a higher energy, or shorter wavelength, than the emitted light. In some embodiments, a dichroic mirror or bandpass or longpass filter can optionally be used to reduce the scattered excitation light before the signal is recorded.

In some embodiments, the detection of G6PD enzyme activity can be performed by an epifluorescence approach in which a dichroic beamsplitter and high numerical aperture lens are used to effectively shorten the pathlength optically rather than requiring a more sensitive detector (compared to the detector sensitivity required for measuring absorbance of a highly diluted sample in a standard cuvette) or a short pathlength disposable assay tube, cuvette or the like. The additional optics increase the instrument cost only a small amount, as compared to an instrument test based on absorption, and this approach does not impart stringent short path length requirements on the disposable assay tube, reducing the cost of the disposable as compared to a short-pathlength disposable. Additionally, this epifluorescence approach allows measuring G6PD enzyme activity in blood at concentrations of up to 60%, 65%, 68%, 70%, 75%, 80%, 85%, 90%, 95% (this refers to the concentration of blood in the final assay—for example, a blood concentration of 68% corresponds to 680 μL , of whole blood per 1000 μL of total assay volume) and, in some instances, higher concentrations. This is particularly beneficial as higher concentrations can increase the concentration of target present for immunoassays as well as provide a simplified workflow by reducing the requirements for sample dilution.

In some embodiments, the detection of G6PD enzyme activity can be performed by an epifluorescence approach in which an instrument which utilizes a LED for illumination, a dichroic beamsplitter to separate excitation and emission light, a lens to focus excitation light on the sample and collect emission light from the sample, and a standard silicon photodiode detector. Additionally, in some embodiments, the instrument would contain a temperature monitor.

In addition or alternatively, in some embodiments, the detection of G6PD enzyme activity can be performed via an Attenuated Total Reflectance (ATR) approach. In the ATR approach, the incident light is reflected at least once or alternatively multiple times at the interface of the assay container and the assay solution. When this occurs, some incident light is transmitted through the interface into the assay solution in the form of an evanescent wave. Configuring such that light undergoes multiple reflections between the tube and assay solution interface increases the number of times the excitation light interacts with the sample, potentially increasing the measured fluorescence signal. Like epifluorescence, these approaches can be used to obtain a very short path length using optical means, the exact length of which is determined by the wavelength of light, refractive indices of the assay tube and assay solution, and the angle of incidence of the excitation light. These approaches can also be used to measure absorbance rather than fluorescence signals.

In some embodiments, the detection of G6PD activity in a blood sample includes measuring a signal that directly or indirectly corresponds to the enzymatic conversion of NADP⁺ to NADPH in the blood sample, preferably where the sample is undiluted, or minimally diluted. The term “signal” as used herein can refer to any detectable parameter.

### C. Bloodborne Microorganisms

In some embodiments, the methods and compositions for detecting G6PD activity in the blood sample, which is preferably undiluted or minimally diluted, disclosed herein can be performed as part of or in conjunction with a diagnostic method for detecting a bloodborne microorganism in the blood sample. In some embodiments, the detection of a bloodborne microorganism is performed on a blood sample that has been diluted. In some embodiments, the detection of a bloodborne microorganism is performed on a blood sample that has not been diluted, i.e. undiluted, or is minimally diluted. In principle, the methods and compositions disclosed herein can be deployed for diagnostic detection and identification of any bloodborne microbial species, including, but not limited to, bacteria, protozoa, molds, yeasts, filamentous microfungi, and viruses. The methods and compositions are preferably used with bloodborne microorganisms that are important or interesting for health-related conditions and diseases. A bloodborne microorganism, as used herein, is one that can be spread through contamination by blood and other body fluids. In some embodiments, the compositions and methods disclosed herein can preferably be used in detecting microbial species that are not usually transmitted directly by blood contact, but rather by insect or other vector, and therefore are also classified as vector-borne microorganisms, even though the causative agents can be found in blood. Non-limiting examples of vector-borne microorganisms include West Nile virus and malaria. Many bloodborne microorganisms can also be transmitted by other means, including transplacental transmission, high-risk sexual activities, or intravenous drug use.

Accordingly, in some embodiments, the compositions and methods of detecting G6PD enzyme activity in blood samples as disclosed herein can be used as part of or in conjunction with the detection and/or identification of one or more bloodborne pathogenic microorganisms in the blood samples. Non-limiting examples of bloodborne pathogenic microorganisms that can be suitably detected include, for instance, any microorganisms from a genus including, but not limited to hepatitis B virus (HBV), hepatitis C virus (HCV), human immunodeficiency virus (HIV), West Nile virus, causative microorganisms of malaria, Dengue fever, Typhoid fever, and syphilis.

In some embodiments, the bacterial pathogenic species detected and/or identified are within the genera including, but are not limited to any Bacillus species including Bacillus anthracis and Bacillus cereus; any Streptococcus species including Streptococcus pneumonia, Streptococcus pyogenes, Streptococcus agalactiae, Streptococcus oralis, Streptococcus mitis; any Staphylococcus species, including Staphylococcus aureus, Staphylococcus epidermidis, and Staphylococcus staphylococci; any Serratia species, including Serratia marcescens; any Klebsiella/Enterobacter species, including Enterococcus faecalis, Klebsiella pneumonia, Enterobacter cloacae, Enterococcus faecium, and Enterobacter aerogenes. Species of Listeria monocytogenes, Escherichia coli, Haemophilus influenzae, Pseudomonas aeruginosa, Acinetobacter battmannii, Neisseria meningitidis, Bacteroides fragilis, Salmonella Typhi, Salmonella enterica, Yersinia pestis, Francisella tularensis, and Brucella abortus are also suitable

In some embodiments, the compositions and methods disclosed herein are preferably used for detecting and/or identifying blood-borne organisms within the genera of Anaplasma, Babesia, Bartonella, Ehrlichia, Leishmania, Mycoplasma, and Rickettsia. Exemplary blood-borne organisms suitable for the methods, compositions, and kits of the present disclosure include, but not limited to, Anaplasma phagocytophilum, Borrelia burgdorferi, Bartonella henselae, Bartonella washoensis, Ehrlihicha canis, Ixodes scapularis, Ixodes pacificus, Rickettsia rickettsii. Further exemplary bacterial pathogenic species detected and/or identified are protozoan parasites like Trypanosoma sp., such as Trypanosoma cruzi, causing Chagas disease, or American sleeping sickness, and Trypanosoma brucei causing African trypanosomiasis.

In some embodiments, the compositions and methods disclosed herein are preferably used for detecting and/or identifying causative microorganisms of malaria. Particularly preferred are malaria causative microorganisms belonging to the parasitic protozoan genera of Plasmodium, Polychromophilus, Rayella, and Saurocytozoon. In some embodiments, Plasmodium species particularly suitable for the methods and compositions disclosed herein include P. brasilianum, P. cynomolgi, P. cynomolgi bastianellii, P. eylesi, P. falciparum, P. inui, P. knowlesi, P. osmani, P. ovale, P. rhodiani, P. schweitzi, P. semiovale, P. shortii, P. simium, and P. vivax. In some particularly preferred embodiments, the causative microorganism of malaria is selected from the group consisting of Plasmodium falciparum, Plasmodium ovale, Plasmodium knowlesi, Plasmodium ovale, and Plasmodium vivax.

### D. Immunoassays

In some embodiments, the detection of G6PD activity and detection of a bloodborne microorganism in blood samples can be carried out by one or more of immunoassay techniques. Antibodies that specifically bind the foregoing pathogenic microorganisms are well known in the art, are commercially available, or can be produced using any one of the methods known in the art. See, for example, Harlow et al., supra, 1988; and Harlow et al., supra, 1999. Antibodies that specifically bind a bloodborne pathogenic microorganism are well known in the art and include, but are not limited to monoclonal antibodies, polyclonal antibodies; human antibodies, humanized antibodies, fragments of antibodies, such as Fab fragments, F(ab)2 fragments, Fv fragments, scFv fragment, synthetic antibodies, and the like. In addition, a large number of antibodies that specifically bind NADPH are known in the art, and are commercially available (from, e.g., Abbexa, Biorbyt, St John's Laboratory).

In some embodiments, a number of immunoassays relying on particles, for example magnetic particles, are particularly suitable for use in diagnostic detection of microbial species according to the methods, compositions, and kits disclosed herein. Microparticle-based immunoassays generally fall into two main categories: homogeneous (separation-free) and heterogeneous assays.

In some embodiments, the detection of G6PD activity and/or detection of a bloodborne microorganism are carried out in a homogeneous (separation-free) assay format, in which binding reactants are mixed and measured without any subsequent washing step prior to detection. The advantages of such a system are fast solution-phase kinetics, a simple assay format, simpler instrumentation as well as lower costs because of fewer assay steps, low volumes and low waste. Homogeneous immunoassays do not require physical separation of bound and free analyte and thus may be faster and easier to perform then heterogeneous immunoassays. Homogeneous immunoassay systems using small sample size, low reagent volume and short incubation times, provide fast turnaround time. Homogeneous assays are the preferred assay format in high throughput screening platforms such as AlphaScreen, SPA, fluorescent polarization and flow cytometry based assays, as well as in diagnostic assays such as particle agglutination assays with nephelometry or turbidimetry as the detection methods.

One of ordinary skill in the art will readily appreciate that one or more microorganisms can be detected and/or identified within a single blood sample. In some embodiments, the detection of two or more microorganisms is carried out sequentially in the same sample. In some embodiments, the detection of two or more microorganisms is carried out in parallel in the same sample.

In some embodiments, the methods disclosed herein comprise homogenous immunoassay techniques employing optically active indicator particles, such as Surface Enhanced Raman Scattering (SERS)-active nanoparticles. Raman scattering is an optical phenomenon in which excitation light generates a fingerprint-like vibrational spectrum of a molecule with features that are much narrower than typical fluorescence spectra. Raman scattering can be excited using monochromatic or nearly monochromatic far-red or near-IR light, photon energies which are typically too low to excite the inherent background fluorescence in biological samples. Since Raman spectra typically cover vibrational energies from 300-3500 cm⁻', it could be possible to measure and distinguish a dozen (or more) tags in a single measurement using a single light source. Additionally, in SERS, molecules in very close proximity to nanoscale roughness features on noble metal surfaces (gold, silver copper) give rise to million- to trillion-fold increases, known as enhancement factor (EF), in scattering efficiency. Further information regarding suitable methods, systems, and devices of SERS-nannotag assays for detection of microorganisms in various types of sample can be found in, for example, Mulvaney et al. Langmuir 19:4784-4790, 2003; Modern Techniques in Raman Spectroscopy, John Wiley & Sons Ltd, Chichester, 1996; Analytical Applications of Raman Spectroscopy, Blackwell Science Ltd, Malden, Mass. 1999; PCT Patent Publication No. WO 2013165615A2, US Patent Publication No. 20120164624A1, and US Pat. No. 6,514,767, the contents of which are incorporated by reference herein in their entireties. Typically, each of the SERS-nanoparticles is associated with one or more specific binding members such as, for example antibodies, having an affinity for one or more microorganisms of interest, and therefore can form a complex with specific microorganisms in the blood samples. Thus, the optically active indicator particles can be any particle capable of producing an optical signal that can be detected in a blood sample without wash steps. Further, magnetic capture particles, also having associated therewith one or more specific binding members having an affinity for the one or more microorganisms of interest, which can be the same or different from the specific binding members associated with the indicator particles, can be used to capture the microorganism-indicator particle complex and concentrate the complex in a localized area of an assay vessel for subsequent detection. In some embodiments, “real-time” detection and identification of microorganisms can be carried out in a sample in which active growth of the microorganism is occurring. In some embodiments, the homogenous immunoassay can be conducted in a biocontained manner without exposure of the user or environment to the sample (“closed system”) and can provide automated, around the clock, detection and identification of microorganisms by monitoring the assay signal over time as the culture progresses. The combination of detection and identification with microbiological culture can lead to earlier availability of actionable results.

In some embodiments of the methods disclosed herein, a capture particle is conjugated with an antibody to capture the antigen of interest such as, for example a pLDH antigen, from the blood sample. The detection particle can be a SERS-nanotag particle as described herein also conjugated with a detection antibody with binding affinity for the antigen of interest, for instance the pLDH antigen. The SERS nanotag includes a Raman reporter molecule as described herein. In the presence of the antigen of interest both the capture particle and the detection particle are bound to form a SERS active, immune-complex comprised of one or more capture particles and one or more detector particles. Homogeneous immunoassays using SERS nanotags provide at least three intrinsic advantages as detection tags. (1) They can be excited in the near-IR, and thus are compatible with whole blood measurement. (2) SERS nanotags generally minimize photobleaching which allows for higher laser powers and longer data acquisition times, resulting in more sensitive measurements. (3) A large number of distinct tags currently exist, enabling highly multiplexed assays.

In some embodiments, the detection of bloodborne microorganisms according to the present disclosure can be performed either directly or indirectly. For direct detection of microorganisms growing in culture, the specific binding members associated with the magnetic capture particles and indicator particles can have an affinity for the largely intact microorganism, e.g. by binding to the surface of the microorganisms. For indirect detection, the binding members associated with the magnetic capture particles and indicator particles may have an affinity for byproducts of the microorganism. Examples of byproducts could include but are not limited to secreted proteins, toxins, and cell wall components. In some embodiments, direct and indirect detection modes may be used alone and/or independently. In some embodiments, direct and indirect detection modes may be used in combination.

In addition or alternatively, the detection of G6PD activity and/or detection of a bloodborne microorganism can be carried out in a heterogeneous immunoassay format, which requires the separation of free analyte and of unbound detector and in certain instances may be more versatile than homogeneous assays. The wash or physical separation steps eliminate most interfering substances and in general do not interfere with the detection/quantification step. Stepwise heterogeneous assays are possible which allow for larger sample size, which in turn improves sensitivity and yields wider dynamic range than the standard assay curves. Methods, systems, reagents, and devices useful for detection of microorganisms in a heterogeneous immunoassay format are known in the art. Many currently available clinical analyzers use magnetic microparticles for heterogeneous diagnostic assays to selectively bind and then separate the analyte of interest from its surrounding matrix using a magnetic field (The Immunoassay Handbook, Nature Publ., London, 2001). Exemplary analyzers based on this format include the ACS-180® and Bayer Immuno 1™ from Bayer Diagnostics, the Access® from Beckman Coulter, and the Elecsys® from Roche Diagnostics.

### E. Biomarkers

Embodiments disclosed herein relate to a method for detecting G6PD activity in a blood sample, preferably an undiluted or minimally diluted blood sample, wherein the G6PD detection is performed as part of or in conjunction with a diagnostic method for detecting a bloodborne microorganism in the blood sample. In a preferred embodiment, both the assay for G6PD activity and the diagnostic method for detecting a bloodborne microorganism are conducted in a blood sample that is undiluted or minimally diluted.

The present disclosure provides for the use of one or more biomarker(s) to give an indication of the malarial status in an individual and/or which may also be used to assess the type of treatment that is appropriate for such an individual. In some embodiments, the at least one biomarker is an antigen of the bloodborne microorganism selected from the group consisting of hypoxanthine phosphoribosyltransferase (pHPRT), phosphoglycerate mutase (pPGM), 14-3-3 protein, heat shock protein 86, heat shock 70kDa protein, QF 122 antigen, enolase, ribosomal phosphoprotein P0, vacuolar ATP synthase catalytic subunit a, elongation factor 1 alpha, proliferating cell nuclear antigen, ribonucleoside-diphosphate reductase large subunit, triose-phosphate isomerise, glyceraldehyde-3-phosphate dehydrogenase, Rab 1 , heat shock protein signal peptide, PfmpC 1 ™ helix, high mobility group protein, chaperonin cpn60 mitochondrial precursor and actin. In a preferred embodiment, the biomarker is selected from the group consisting of lactate dehydrogenase, histidine rich protein II, and pHPRT,

In some embodiments, preferably the presence of any of the biomarkers is indicative/diagnostic of malaria in a subject. In some embodiments, the methods disclosed herein may include a further step of concluding a subject has malaria if one or more of the biomarkers are found present in the blood sample. In some embodiments, the methods disclosed herein may further comprise a step of comparing the level of the one or more of the biomarkers with one or more pre-determined reference values. In some embodiments, the level of one or more of the biomarkers may be compared to the level in a control sample, preferably a non-infected sample, to allow for any inaccuracy or background in the test method used.

The methods disclosed herein may be used, for example, for any one or more of the following: to diagnose malaria; to advise on the prognosis of a subject with malaria; to monitor disease progression; and to monitor effectiveness or response of a subject to a particular treatment.

The integrated method for detecting G6PD activity in blood samples, preferably a sample which is undiluted or minimally diluted, in combination with detection of malaria as disclosed herein may be used in conjunction with an assessment of clinical symptoms to provide a more effective diagnosis of malaria.

### F. Kits

The methods disclosed herein may be performed, for example, by utilizing kits. The present disclosure also relates to kits for detecting an amount of glucose-6-phosphate dehydrogenase (G6PD) activity in a blood sample, preferably wherein the sample is undiluted or minimally diluted. The kit, in some embodiments, includes reagents needed for performing one or more methods disclosed herein in suitable packaging, and optionally written material that can include one or more of the following: instructions for use, discussion of clinical studies, listing of side effects, and the like. In some embodiments, the kits includes glucose-6-phosphate (G6P) or a G6P surrogate; nicotinamide adenine dinucleotide phosphate (NADP⁺); and optionally instructions for preparing a reaction mixture that facilitates a reaction of NADP', G6P, and G6PD in a blood sample, preferably in an undiluted or minimally diluted blood sample. In some embodiments, the kits can further include a surfactant and a buffer for preparing a reaction mixture that facilitates the reaction of NADI)⁺, G6P and G6PD. In some embodiments, the kits can further include an enzyme denaturant. In some embodiments, the kits further include instructions for preparing a sample for analysis using epifluorescence spectroscopy or detection. In further embodiments, the kits also include a calibration curve which comprises a plot of NADPH concentration versus absorbance or fluorescence signals. In preferred embodiments, the components of the kits are adapted for use in an undiluted or minimally diluted blood sample. Adaptation for use in an undiluted or minimally diluted blood sample can include increased concentrations or volumes of G6P and NADP⁺ reagents, buffer components such as lysing or stabilizing agents, and the like.

The buffer and/or the surfactant may be provided in a container in dry form or liquid form. Exemplary buffers and surfactants suitable for measuring G6PD activity are known in the art and are provided herein in the Examples. The buffer is typically present in the kits at least in an amount sufficient to produce a particular pH in the mixture. In some embodiments, the buffer is provided as a stock solution having a pre-selected pH and buffer concentration. In some embodiments, acids and/or bases are also provided in the kits in order to adjust the reaction mixture to a desired pH. The kits may additionally include one or more one or more diluents, e.g., solvents suitable for use in one or more of the methods disclosed herein. The kits may additionally include other components that are beneficial to enzyme activity, such as salts (e.g., KCl, NaCl, or NaOAc), metal salts (e.g., Ca₂⁺ salts such as CaCl₂, MgCl₂, MnCl₂, ZnCl₂, or Zn(OAc), and/or other components that may be useful for the G6PDH enzyme. These other components can be provided separately from each other or mixed together in dry or liquid form. In some embodiments, the kit further includes a disposable cuvette. In some embodiments, the cuvette is prefilled with suitable reagents, including one or more of the following NADP⁺, G6PD, NaCl, Tris, Triton-X, EDTA, and/or other buffer, cell lysing reagents, or stabilizing components. In preferred embodiments, the components of the kits are adapted for use in an undiluted or minimally diluted blood sample as disclosed herein.

The reagents, for example the glucose-6-phosphate (G6P) and/or nicotinamide adenine dinucleotide phosphate (NADP), can be provided in dry or liquid form, together with or separate from the buffer. To facilitate dissolution in the reaction mixture, the reagents (e.g. G6P and/or NADP) can be provided in an aqueous solution, partially aqueous solution, or non-aqueous stock solution that is miscible with the other components of the reaction mixture. In preferred embodiments, the components of the kits are adapted for use in an undiluted or minimally diluted blood sample as disclosed herein.

In some embodiments, the kits further include instructions for use. For example, the kit can include instructions for preparing a reaction mixture that facilitates a reaction of NADI)⁺, G6P and G6PD enzyme. In some embodiments, the kits comprise a pre-prepared calibration plot which would allow a user to determine the G6PD activity in the sample based upon an amount or rate of production of NADPH determined using epifluorescence spectroscopy and/or absorbance measurements for detection. For example, the pre-prepared calibration plot may be a plot of NADPH concentrations versus fluorescence or absorbance signal and/or G6PD activity level. In some embodiments, the kit includes standard samples, with pre-determined amounts of G6PD, such that a user may produce their own calibration plot. In some embodiments, the kit further includes instructions on how to prepare a calibration plot. In preferred embodiments, the instructions, information, and/or calibration curves are adapted for use in an undiluted or minimally diluted blood sample as disclosed herein.

### EXAMPLES

Additional embodiments are disclosed in further detail in the following examples, which are not in any way intended to limit the scope of this disclosure or the claims.

### Example 1

SERS Homogenous No Wash Assay and G6PD Assay on Undiluted Blood Samples

This Example describes experiments detecting G6PD enzymatic activity in undiluted blood samples that were performed in conjunction with a diagnostic assay of Plasmodium vivax, which is a bloodborne causative pathogen of malaria.

Blood samples: Undiluted blood samples from G6PD normal or G6PD deficient subjects were spiked with 0 ng/mL or 150 ng/mL of a lactate dehydrogenase (pLDH) antigen of Plasmodium vivax. For each sample, G6PD and HNW reagents were mixed a lysing assay buffer and a human blood substitute sample (Trinity Biotech) that was spiked with either 0 or 150 ng/mL of of P. vivax recombinant LDH antigen. The LDH antigen was added to the assay mixture to bring the concentration to 9% blood.

Homogenous No-Wash (HNW) Assays: Homogenous no-wash (HNW) reagents were prepared as follows: SERS tags were conjugated to anti-P. vivax LDH antibodies, and 1 micron magnetic beads were conjugated to anti-panLDH antibodies using standard conjugation chemistry techniques.

Each of the sample mixtures was incubated for 20-30 minutes. Subsequently, the magnetic beads (and any bead-antigen-SERS tag immune complexes) were pelleted using a magnetic rack, and the pellet was interrogated with a near-infrared laser using a HNW assay measurement instrument.

G6PD Assay: Following the HNW assays described above, the supernatant (i.e. the sample with the exclusion of the magnetic bead pellet) of each of the sample mixtures was collected, placed in a cuvette (VWR 47743-836, Brandtech 759240, or equivalent), and the G6PD enzyme activity was monitored by measuring the NADPH production for 5 minutes via the UV/Visible absorbance at 315 nm, making use of the spectrophotometric change when NADPH is produced. In these experiments, G6PD reagent solutions were prepared from stock solutions of Nicotinamide Adenine Dinucleotide Phosphate (NADI)⁺, Sigma Aldrich, Catalogue No. N8160-15V) and Glucose-6-phosphate (G6P, Sigma Aldrich, Catalogue No. G7879), in accordance with manufacturers' recommendations. When testing clinical samples, 1 bottle of NADP' typically provided enough reagent to test 2 clinical samples. The content of each bottle of NADP⁺ was rehydrated by adding to the bottle 200₁11 of an assay buffer (which contained one or more suitable stabilizing agents, lysing agents, pH buffering agents, and salts), followed by swirling and tilting the bottle for 30 seconds to ensure that the NADP⁺ was thoroughly rehydrated. The rehydrated NADP⁺ is typically stable at room temperature for up to 3 hours. The buffer composition included effective concentrations of sodium chloride, Tris, Triton-X-100, Bovine Serum Albumin, sodium azide.

The G6PD enzyme activity hemolyzed blood normal controls used in these experiments were G6PD normal enzyme activity control (G5888, Trinity Biotech), and G6PD deficient enzyme activity control (G6888, Trinity Biotech). Each of the Trinity enzyme activity controls were rehydrated by addition of 0.5 mL of water. The samples were gently swirled for 10 seconds and allowed 5 minutes for rehydration.

For each reaction in a microfuge tube, the following reagents were added: 300 μL of assay buffer to a fresh microfuge tube, 78 μL of NADP+, 78 μL of G6P stock solution to the microfuge tube, and 45 μL of rehydrated Trinity enzyme activity control. The microfuge tube was capped and vortexed for 1-2 seconds to mix thoroughly. The content of the microfuge tube was then transferred into a disposable cuvette (VWR 47743-842, Brandtech 759240, or equivalent), and the cuvette was placed into the epifluorescence instrument ensuring that the beaker symbol on the cuvette was facing the incident beam. As the G6PD enzyme /NADP+ reaction started upon mixing, the reagents were not mixed until immediately prior to data collection. The cuvette was removed from the instrument and discarded when the assay was complete.

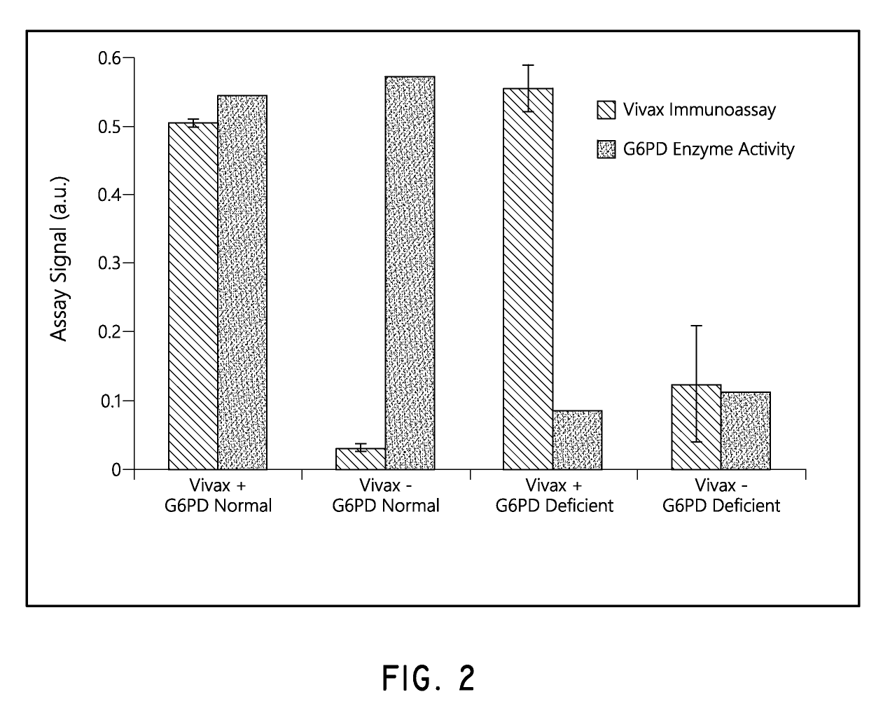

The results of the G6PD assays and the malaria detection assays performed on the same blood samples are summarized in FIG. 2. Levels of G6PD enzymatic activity in the presence (150 ng/mL; Vivax+) or absence (0 ng/mL; Vivax−) of the pLDH) antigen are indicated by solid bars. Malaria detection immunoassays, which were performed using a monoclonal antibody specific to the P. vivax pLDH antigen, are indicated by dashed bars.

### Example 2

### Detection of G6PD Activity by Monitoring NADPH Production in Undiluted Blood Samples

This Example describes experiments detecting G6PD activity in minimally diluted blood samples by monitoring NADPH production.

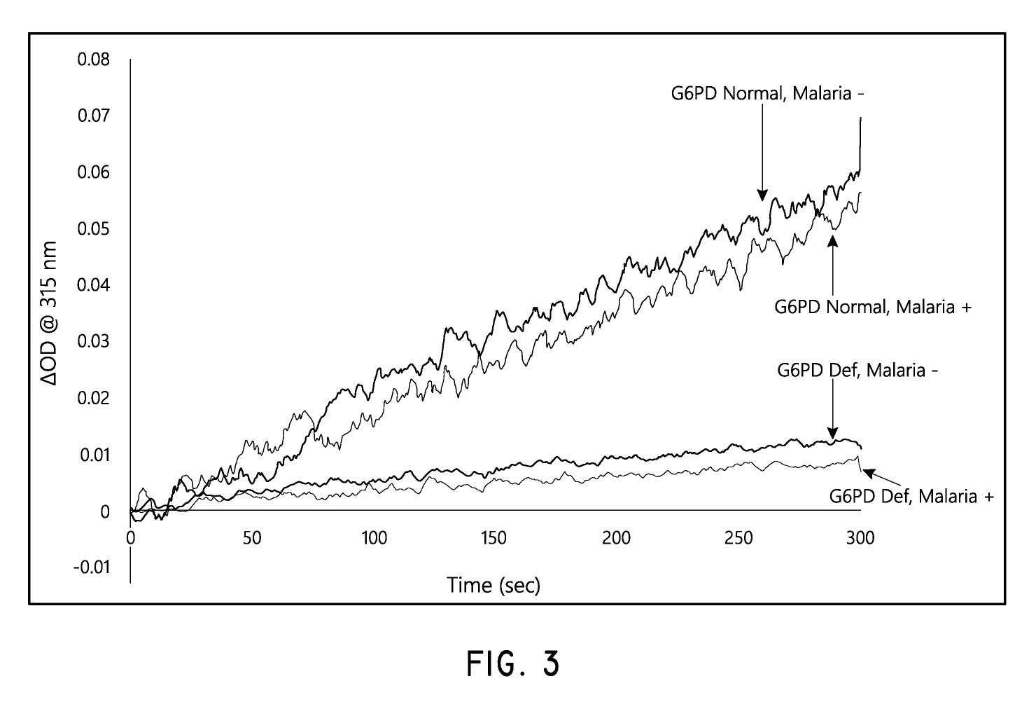

In these experiments, all assays were run at 9% blood (i.e. 90 μL of blood per 1000 μL of total assay volume). Following the HNW assays described in Example 1, the supernatant (i.e. the sample with the exclusion of the magnetic bead pellet) of each of the sample mixtures was collected, placed in a cuvette (VWR 47743-836, Brandtech 759240, or equivalent), and the G6PD enzyme activity was monitored by measuring the NADPH production for 5 minutes via the UV/Visible absorbance at 315 nm, making use of the spectrophotometric change when NADPH is produced. Absorption was monitored at 315 nm rather than 340 nm because the optical density was too high to read at the 340 nm NADPH absorbance peak. By reading at 315 nm, which corresponds to the “shoulder” of the absorbance peak, the NADPH absorbance was more accurately measured. The results of experiments measuring levels of G6PD enzymatic activity in the sample mixtures are graphically summarized in FIG. 3. Undiluted blood samples from G6PD normal (G6PD Normal) or G6PD deficient (G6PD Def) subjects were spiked with 0 ng/mL or 150 ng/mL of a lactate dehydrogenase (pLDH) antigen of Plasmodium vivax. Levels of G6PD enzymatic activity were assayed in the presence (150 ng/mL; Malaria+) or absence (0 ng/mL; Malaria−) of the pLDH antigen. The 315 nm absorbance was monitored for 5 minutes, and the change in OD over this time period was calculated. “A.U.”: Arbitrary Unit.

### Example 3

Epifluorescent Detection of G6PD Activity in Different Blood Concentrations

This Examples describes the use of epifluorescence microscopy techniques for the detection of G6PD enzymatic activity in three different blood concentrations, demonstrating that the epifluorescence G6PD enzymatic assays can be reliably performed at a wide range of blood loads. In these experiments, G6PD assays were performed as described in Example 1 above.

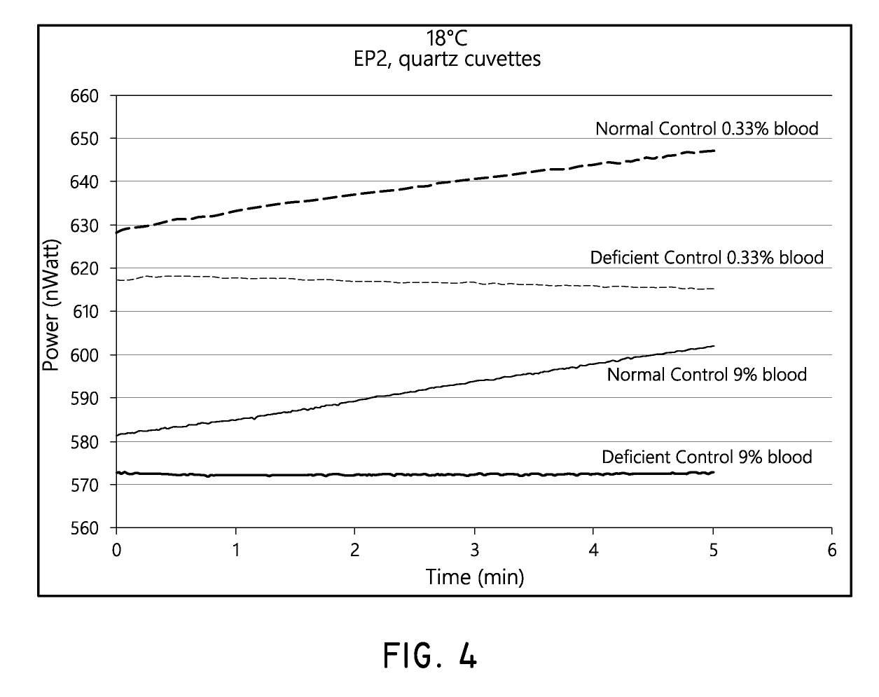

In one experiment, G6PD enzyme activity of normal and deficient hemolyzed blood controls (Trinity Biotech) were tested in the epifluorescence instrument at blood loads of 9% and 0.33%. Reagents concentrations were scaled proportionally the blood concentration. The plots of FIG. 4 graphically summarize epifluorescence G6PD enzymatic data of blood samples from normal and deficient hemolysate blood controls. It was observed that the slopes were nearly identical at both blood concentrations, demonstrating the presently disclosed method can be used at a wide range of blood concentrations.

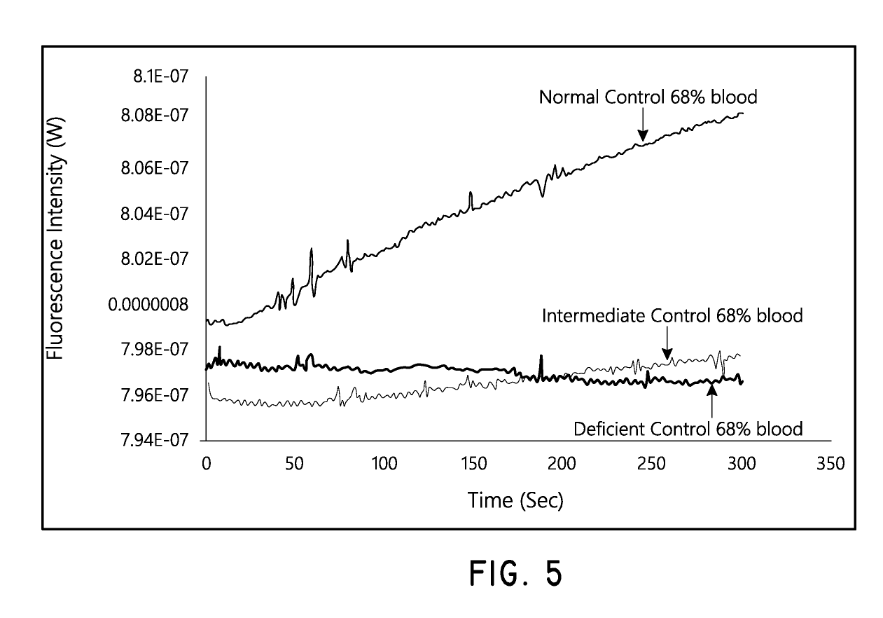

In another experiment, G6PD enzymatic activity was measured in three hemolyzed blood controls: Normal, Intermediate, and Deficient (Trinity Biotech) epifluorescence instrument at blood loads of 68% using the epifluorescence method described in Example 1. Assay reagent concentrations were increased to ensure that enzyme activity rather than reagent concentration was the limiting factor for the reduction of NADP+to NADPH. As shown in FIG. 5, when blood concentrations were as high as 68%, the epifluorescence assay method disclosed herein could still clearly distinguish the Normal blood sample from remaining two samples, Intermediate and Deficient.

### Example 4

Epifluorescence Detection of G6PD Activity at Different Temperatures

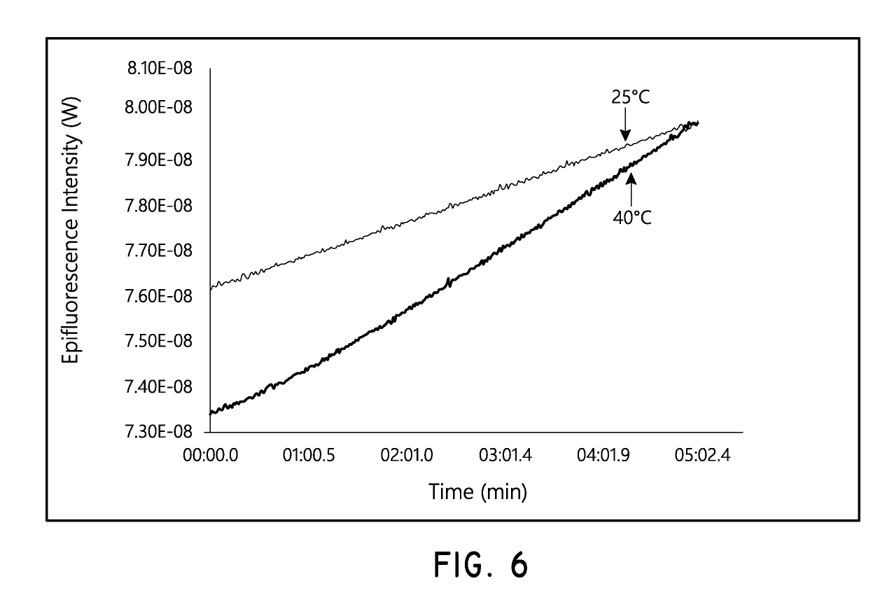

This Example describes experiments measuring levels of G6PD enzymatic activity by epifluorescence detection at two different temperatures, illustrating that the epifluorescence approach disclosed herein can be deployed for a wide temperature range. In this experiment, G6PD enzyme activity of a normal enzyme activity blood sample was tested at 18° C. and 40° C. by placing the epifluorescence instrument in an incubator with heating and cooling capability. The experiments presented in FIG. 6 were performed at 25° C. and 40° C. while the experiments presented at FIG. 2 above were performed at 18° C. It is observed that the slope was dependent upon temperature, which was consistent with the previously reported finding that G6PD enzymatic activity is temperature dependent.

### Example 5

### Epifluorescence Detection of G6PD Activity and Hemoglobin Measurement of Undiluted Blood Samples

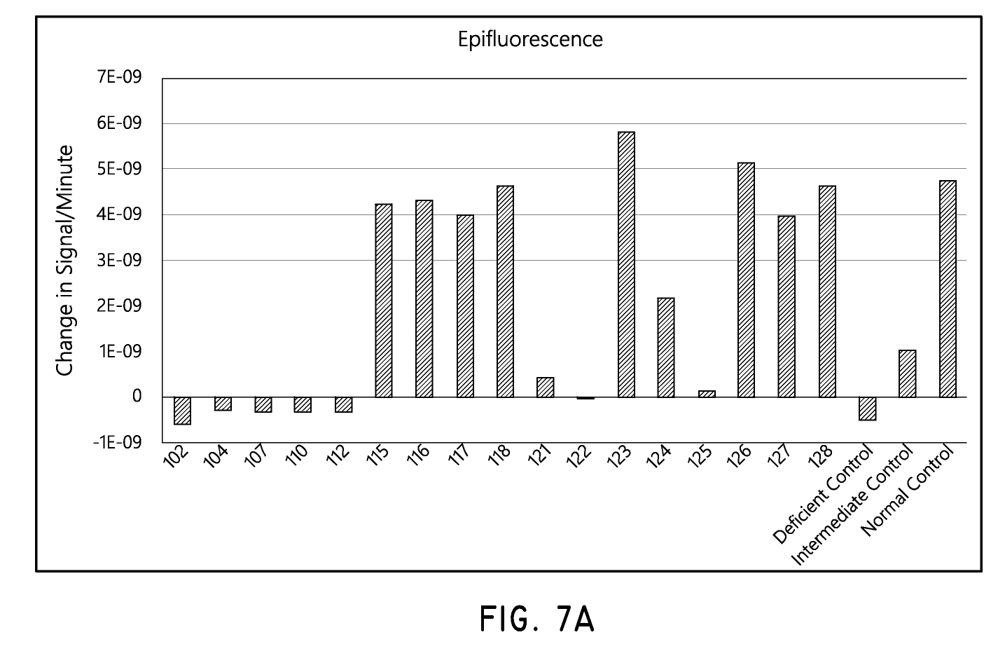

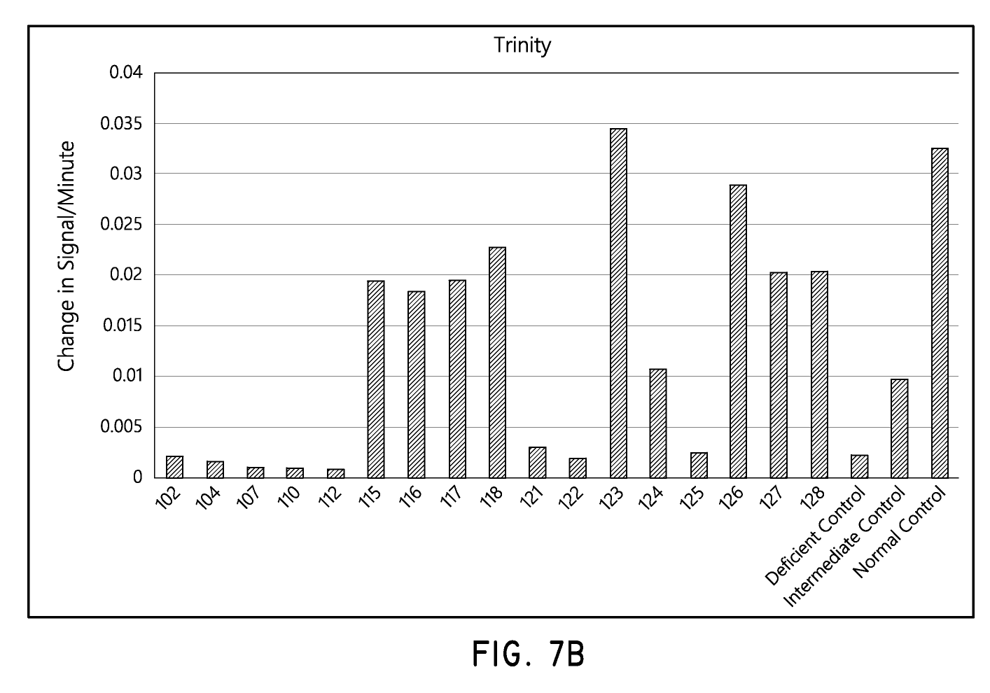

This Example describes experiments measuring levels of G6PD enzymatic activity in large numbers of undiluted clinical blood samples. In these experiments, anticoagulated blood samples were procured from febrile patients in Thailand and assayed for G6PD activity via an epifluorescence detection procedure according to the methods disclose herein (FIG. 7A) or by a commercial Trinity Quantitative Kit (FIG. 7B).

In these experiments, G6PD assays were performed as described in Example 1 above. G6PD reagent solutions were prepared from stock solutions of NADP⁻ and G6P (Sigma Aldrich) in accordance with manufacturer's recommendations. When testing clinical samples, 1 bottle of NADP+ typically provided enough reagents to test 2 clinical samples. The content of each bottle of NADP+ was rehydrated by adding 200 μL of assay buffer (which contained one or more suitable stabilizing agents, lysing agents, pH buffering agents, and salts to the bottle, followed by swirling and tilting the bottle for 30 seconds to ensure that the NADP⁺ was thoroughly rehydrated. The rehydrated NADP' is typically stable at room temperature for up to 3 hours. Each clinical sample was vortexed for approximately 5 seconds or until well mixed. In a microfuge tube, the following reagents were added: 300 μL of assay buffer containing effective concentrations of sodium chloride, Tris, Triton-X, Bovine Serum Albumin, and sodium azide, 78 μL of NADP solution, 78 μL of G6P solution, and 45 μL of blood sample. The content of the microfuge tube was then transferred into a disposable cuvette (VWR 47743-842, Brandtech 759240, or equivalent), and the cuvette was placed into the epifluorescence instrument ensuring that the beaker symbol on the cuvette was facing the incident beam. As the G6PD enzyme /NADP⁻ reaction started upon mixing, the reagents were not mixed until immediately prior to data collection. The cuvette was removed from the instrument and discarded when the assay was complete.

FIG. 7 summarizes the results of experiments measuring levels of G6PD enzymatic activity in 17 undiluted clinical blood samples. Also included in this experiment were three G6PD enzymatic activity controls: G6PD normal enzyme activity control (G5888, Trinity Biotech), G6PD Intermediate Enzyme Activity Control (Trinity Biotech G5029), and G6PD deficient enzyme activity control (G6888, Trinity Biotech). Each of the Trinity enzyme activity controls were rehydrated by addition of 0.5 mL of water. The samples were gently swirled for 10 seconds and allowed 5 minutes for rehydration. Trinity Quantitative assays were performed in accordance with the manufacturer's recommendations with minor modifications. HemoCue Hb 201⁺ (HemoCue, Inc., Lake Forest, Calif.) assays were performed to measure the hemoglobin measurement of each sample, according to the manufacture's recommended instructions.

Negative values observed in the epifluorescence data were determined to be due to photobleaching of the plastic cuvettes used for testing, rather than a biological phenomenon.

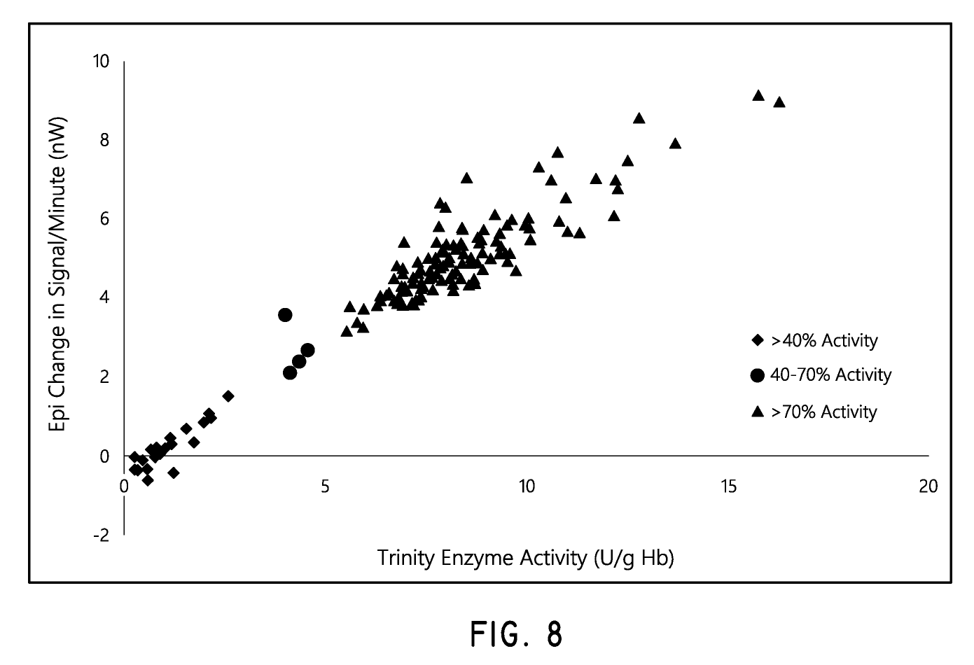

FIG. 8 is a graphical representation of Trinity quantitative test and epifluorescence test concordance from testing of 154 undiluted blood samples. It was observed that the epifluorescence test, which did not include a separate hemoglobin (“Hb”) measurement, correctly classified 153 of the 154 samples into the correct enzyme activity range. Negative values observed in the epifluorescence data are due to photobleaching of the plastic cuvettes used for testing, rather than a biological phenomenon. In the Trinity quantitative tests, the G6PD activity was normalized relative to the reported mean enzyme activity of a healthy adult male, which was 7.17 IU/g Hb.

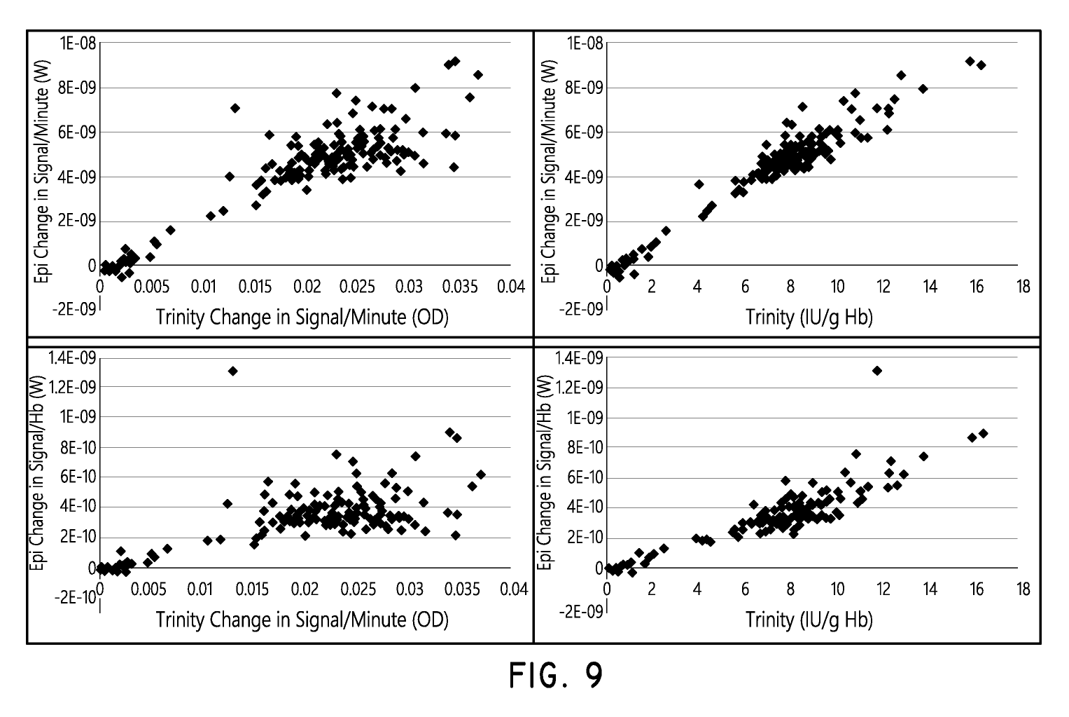

FIG. 9 is a graphical representation of Trinity quantitative test and epifluorescence test concordance from testing of 154 undiluted blood samples. Each of the samples was assayed for G6PD activity by (1) Trinity quantitative test and (2) epifluorescence test according to following four experimental setups. (Top Left) Neither Trinity quantitative test nor epifluorescence test was normalized by a separate hemoglobin measurement; Pearson Correlation Coefficient=0.896. (Top Right) Trinity quantitative test was normalized by a separate hemoglobin measurement, while epifluorescence test was not normalized by a separate hemoglobin measurement; Pearson Correlation Coefficient=0.968. (Bottom Left) Trinity quantitative test was not normalized by a separate hemoglobin measurement, while epifluorescence was test normalized by a separate hemoglobin measurement; Pearson Correlation Coefficient=0.671. (Bottom Right) both Trinity quantitative test and epifluorescence test were normalized by a separate hemoglobin measurement; Pearson Correlation Coefficient=0.893 Pearson Correlation Coefficient=0.935 (with single outlier at 11.73 IU/g Hb removed). Negative values observed in the epifluorescence data were determined to be due to photobleaching of the plastic cuvettes used for testing, rather than a biological phenomenon.

TABLE 1
Pearson correlation coefficients for Trinity and
Epifluorescence testing of 154 clinical samples with
and without hemoglobin measurement normalization

| | Trinity without | Trinity with |
|---|---|---|
| | hemoglobin | hemoglobin |
| | measurement | measurement |
| Epifluorescence | 0.896 | 0.968 |
| without hemoglobin | | |
| measurement | | |
| Epifluorescence | 0.671 | 0.893* |
| with hemoglobin | | |
| measurement | | |

*this correlation coefficient is 0.935 with single outlying point removed.

All of the references disclosed herein, including but not limited to journal articles, textbooks, patents and patent applications, are hereby incorporated by reference for the subject matter discussed herein and in their entireties. Throughout this disclosure, various information sources are referred to and incorporated by reference. The information sources include, for example, scientific journal articles, patent documents, textbooks, and World Wide Web browser-inactive page addresses. The reference to such information sources is solely for the purpose of providing an indication of the general state of the art at the time of filing. While the contents and teachings of each and every one of the information sources can be relied on and used by one of skill in the art to make and use the embodiments disclosed herein, any discussion and comment in a specific information source should no way be considered as an admission that such comment was widely accepted as the general opinion in the field.

The discussion of the general methods given herein is intended for illustrative purposes only. Other alternative embodiments will be apparent to those of skill in the art upon review of this disclosure, and are to be included within the spirit and purview of this application. Although the general methods and compositions have been described with respect to the detailed embodiments thereof, it will be understood by those skilled in the art that various changes in form and detail may be made without departing from the spirit and scope of the disclosure. ndeed, one of the advantages of the presently disclosed methods and compositions is the ability to analyze blood without the need for diluents. That said, in alternative embodiments the methods and compositions disclosed herein can be used on blood that has been diluted for various reasons provided the dilution factor of the sample is known or is determinable.

What is claimed is:

1. A method for detecting glucose-6-phosphate dehydrogenase (G6PD) activity, said method comprising:
a. obtaining an undiluted or a minimally diluted blood sample; and
b. detecting G6PD activity present in said undiluted or minimally diluted blood sample, wherein said detecting G6PD activity comprises performing epifluorescence detection on said undiluted or minimally diluted blood sample.

2. The method of claim 1, wherein said detecting G6PD activity comprises measuring a signal corresponding to the enzymatic conversion of NADP⁺ to NADPH in said undiluted or minimally diluted blood sample.

3. The method of any one of claims 1 to 2, further comprising measuring NADPH fluorescence.

4. The method of any one of claims 1 to 3, wherein said measuring NADPH fluorescence is spectrophotometrically performed via measurement of the NADPH emission when excited by ultraviolet light.

5. The method of any one of claims 3 to 4, where said NADPH fluorescence is excited at a wavelength or wavelengths between 290-400 nm.

6. The method of any one of claims 3 to 5, wherein said NADPH fluorescence is excited at a wavelength or wavelengths between 310-380 nm.

7. The method of any one of claims 3 to 6, wherein said NADPH fluorescence is excited at a wavelength or wavelengths between 330-370 nm.

8. The method of any one of claims 1 to 7, wherein said detecting G6PD activity is performed via an Attenuated Total Reflectance (ATR) approach.

9. The method of any one of claims 1 to 8, wherein said detecting G6PD activity comprises measuring a signal corresponding to the conversion of glucose-6-phosphate (G6P) to 6-phosphoglucono-lactone in said undiluted or minimally diluted blood sample.

10. The method of any one of claims 1 to 9, wherein said detecting G6PD activity in the undiluted or minimally diluted blood sample is performed as part of or in conjunction with a diagnostic method for detecting a bloodborne microorganism in said blood sample.

11. The method of claim 10, wherein said detection of G6PD activity and detection of a bloodborne microorganism are performed on the same aliquot of undiluted or minimally diluted blood sample.

12. The method of any one of claims 10 to 11, wherein said bloodborne microorganism is selected from the group consisting of a bacterium, a protozoan, a mold, a yeast, a filamentous microfungus, and a virus.

13. The method of any one of claims 10 to 12, wherein said bloodborne microorganism is a causative microorganism of malaria.

14. The method of any one of claims 10 to 13, wherein said causative microorganism of malaria is a microorganism belonging to a protozoan genus selected from the group consisting of Plasmodium, Polychromophilus, Rayella, and Saurocytozoon.

15. The method of claim 14, wherein said causative microorganism of malaria is a Plasmodium microorganism belonging to a subgenus selected from the group consisting of Asiamoeba, Bennettinia, Carinamoeba, Giovannolaia, Haemamoeba, Huffia, Lacertamoeba, Laverania, Novyella, Paraplastnodium, Plasmodium, Sauramoeba, and Vinckeia.

16. The method of claim b, wherein said Plasmodium microorganism is selected from the group consisting of Plasmodium falciparum, Plasmodium knowlesi, Plasmodium malariae, Plasmodium ovale, and Plasmodium vivax.

17. The method of any one of claims 10 to 16, wherein said detection of a bloodborne microorganism in said undiluted or minimally diluted blood sample comprises determining the level, or the presence, of at least one biomarker specific to said bloodborne microorganism.

18. The method of claim 17, wherein said at least one biomarker is an antigen specific to said bloodborne microorganism selected from the group consisting of aldolase (pFBPA), histidine-rich protein 2 (HRP-2), hypoxanthine phosphoribosyltransferase (pHPRT), lactate dehydrogenase (pLDH), and phosphoglycerate mutase (pPGM).

19. The method of claim 18, wherein said at least one biomarker is an antigen specific to lactate dehydrogenase (pLDH).

20. The method of any one of claims 10 to 19, wherein said detection of a bloodborne microorganism is carried out by an immunoassay.

21. The method of any one of claim 10 to claim 20, said detection of a bloodborne microorganism is carried out by a sandwich immunoassay.

22. The method of any one of claims 20 to 21, wherein said immunoassay for detection of the bloodborne microorganism is a microparticle-based SERS nanotag immunoassay.

23. The method of claim 22, wherein said microparticle-based SERS nanotag immunoassay for detection of the bloodborne microorganism is a homogenous immunoassay.

24. The method of claims 20 to 21, wherein said immunoassay for detection of the bloodborne microorganism is an Enzyme Linked Immunosorbent Assay.

25. The method of any one of claims 10 to 24, wherein said detection of G6PD activity and detection of a bloodborne microorganism are carried out simultaneously on a single reaction mixture.

26. The method of any one of claims 10 to 24, wherein said detection of G6PD activity and detection of a bloodborne microorganism are carried out sequentially on a single reaction mixture.

27. The method of any one of claims 10 to 24, wherein said undiluted or minimally diluted blood sample is divided into sample aliquots prior to being subjected to said detection of G6PD activity and detection of a bloodborne microorganism.

28. The method of claim 27, wherein said detection of G6PD activity and detection of a bloodborne microorganism are carried out in spatially discrete sample aliquots.

29. A kit for detecting an amount of glucose-6-phosphate dehydrogenase (G6PD) activity in an undiluted or minimally diluted blood sample, said kit comprising:
a. glucose-6-phosphate (G6P) or a G6P surrogate adapted for use in an undiluted or a minimally diluted blood sample; and
b. nicotinamide adenine dinucleotide phosphate (NADP+) or NADP+surrogate adapted for use in an undiluted or a minimally diluted blood sample.

30. The kit of claim 29, further comprising instructions for preparing a reaction mixture that facilitates a reaction of NADP+, G6P, and G6PD enzyme in an undiluted or a minimally diluted blood sample.

31. The kit of any of the preceding claims further comprising immunoassay reagents for detection of a bloodborne microorganism.

32. The method or kit of any of the preceding claims, wherein the undiluted or minimally diluted blood sample is from a subject.

33. The method or kit of claim 32, wherein the subject is suffering from, or suspected of suffering from a disease.

34. The method or kit of claim 33, wherein the subject is suffering from, or suspected of suffering from a febrile illness.

35. The method or kit of claim 33, wherein the disease is caused by a bloodborne microorganism.

36. The method or kit of claim 35, wherein the disease is malaria.

37. The method or kit of any of the preceding claims, wherein in said blood sample subject to analysis the concentration of whole blood or a blood component in the final reaction mixture is greater than 0.1%.

38. The method or kit of any of the preceding claims, wherein in said blood sample subject to analysis the concentration of whole blood or a blood component in the final reaction mixture is greater than 1%.

39. The method or kit of any of the preceding claims, wherein in said blood sample subject to analysis the concentration of whole blood or a blood component in the final reaction mixture is greater than 25%, 30%, 40%, 50%, 60%, 68%, 70%, 80%, or 90%.

40. The method or kit of any of the preceding claims, wherein in said blood sample subject to analysis the concentration of whole blood or a blood component in the final reaction mixture is from 0.1% to 95%, 1% to 95%, 1% to 68%, 25% to 99%, 25% to 95%, 25% to 70%, 40% to 80%, 40% to 68%, 50% to 95%, 50% to 68%, 60% to 95%, 60% to 80%, 68% to 95%, or 68% to 90%.

41. The method or kit of any of the preceding claims, wherein in said blood sample subject to analysis the concentration of whole blood or a blood component in the final mixture is undiluted.
